(19) United States
(12) Patent Application Publication
(10) Pub. No.: US 2014/0107616 A1
(43) Pub. Date: Apr. 17, 2014

(54) Bloodless Arterial Percutaneous Insertion System

(71) Applicant: M. Samy Ahmed Hamboly, 10th of Ramadan City (EG)

(72) Inventor: M. Samy Ahmed Hamboly, 10th of Ramadan City (EG)

(21) Appl. No.: 14/053,107

(22) Filed: Oct. 14, 2013

Related U.S. Application Data
(60) Provisional application No. 61/715,114, filed on Oct. 17, 2012.

(51) Int. Cl. A61M 25/06; A61B 17/34; A61M 39/10

(52) U.S. Cl. CPC A61M 25/0606; A61M 39/10; A61B 17/3403; A61M 25/0693
USPC 604/510; 604/533

(57) ABSTRACT

A needle connector assembly comprises a hemostatic segment and a side tube. The hemostatic segment comprises an opening permitting passage of a guidewire therethrough. The hemostatic segment comprises a proximal portion comprises a perimeter ring and a spanning member. The distal portion comprises a bell or rounded configuration. Upon insertion of a guidewire, the hemostatic segment generally seals around the guidewire such that blood or other fluids are prevented from entering the area proximal to the hemostatic segment. The side tube comprises a closed distal end and is positioned between the proximal end and the distal end of the needle connector assembly. The side tube is in fluid communication with the passageway of the connector assembly and is adapted to receive blood or other fluids. The connector assembly is adapted for connection with a needle assembly.

This application claims the benefit of U.S. provisional application Ser. No. 61/715,114, filed Oct. 17, 2012.

### FIELD OF THE INVENTION

The present invention relates generally to an arterial percutaneous insertion system and specifically to a bloodless arterial percutaneous insertion system comprising a coiled chamber that prevents blood loss after puncturing an artery with a needle, while providing a visual indication to the clinician that the needle has been properly placed within a blood vessel.

### BACKGROUND OF THE INVENTION

Health care providers often find it necessary to infuse liquids or place devices within the veins or arteries of a patient. Such procedures, known as percutaneous entry procedures, include, for example, the placement of intravenous catheter systems (IVs) which are commonly used to administer or draw fluids into and out of a patient's blood vessels. In the typical percutaneous entry procedure, the clinician inserts a needle through the patient's skin and into a vein or artery. Upon entering such vessel, blood is caused to return through a needle cannula towards the clinician. Such blood return causes a “squirt” of blood to be forced out of the proximal end of the needle. Clinicians are trained to consider such squirts of blood as an indication that the needle tip is in the desired position. Once the needle tip is properly positioned, a guidewire may then be inserted through the needle cannula and passed into the blood vessel. Guidewires are used in numerous medical procedures, including cardiac catheterization, interventional radiology, and endovascular surgery. After being inserted through the cannula within the blood vessel, the guidewire is generally advanced to a predetermined point within the vascular system. Guidewires may be passed through arteries to, for example, guide and place arterial catheters. Following insertion of the guidewire, the needle is removed, and a catheter sheath is advanced over the guidewire and into the artery. The guidewire is then removed.

The following will describe the standard manner in which a cardiovascular catheter for diagnostic or treatment purposes may be inserted within a patient. Such a catheter may be placed within the arterial system through a sheath introducer. An arterial sheath should be placed first then followed by the catheters. Percutaneous catheterization of the femoral artery is accomplished using a modified Seldinger technique. The same technique is generally used for both arterial and venous access. The femoral artery approach is the preferred approach for catheterization, although a radial artery approach may also be used. The ideal puncture site is the common femoral artery. Local anesthetic is administered prior to puncture of the artery by a needle. When a pulsating gush of blood comes out of the needle, this is an indication that the needle is inside the artery. A guidewire is then passed through the needle. The needle is then removed with the guidewire remaining within the artery. The sheath is then advanced around the guidewire into the artery. The guidewire is then removed.

The femoral artery sheath is generally 4 to 6 French (1.35-2 mm) and equipped with a valve to prevent any gush of arterial blood. A cardiovascular catheter may then be advanced through the sheath. When the procedure is completed the catheter is removed through the sheath. The sheath remains in situ until anticoagulants are below their peak action.

One common issue resulting from the placement of a needle tip within a blood vessel is the escape of blood from the needle. The patient, the clinician, and other members of the healthcare provider team may then be exposed to this blood which presents significant potential health risks.

The present invention provides a bloodless arterial percutaneous insertion system which prevents the escape of blood through the proximal end of a percutaneous insertion needle but permits the clinician to readily identify that the needle end has entered the blood vessel.

### DESCRIPTION OF THE PRIOR ART

In order to help reduce the risks associated with exposure to blood, there have been efforts to introduce devices that prevent undesirable blood escape during percutaneous entry procedures. Such devices generally are designed to contain the blood that returns from the patient as a result of the needle entering the blood vessel. Some devices comprise “flash chambers” which permit the clinician to visualize the return of blood upon needle insertion within a blood vessel but prevents the blood from escaping from the instrument. Some devices comprise valves that prevent blood from escaping from the needle while still permitting the clinician to insert a guidewire through the needle cannula. For example, Emmert et al. US 2010/0217195 A1 teaches a system comprising a flash chamber and a flash window. Vaillancourt, US 2006/0264834 A1 teaches a valve for mounting on the hub of a percutaneous entry needle to provide a flashback chamber to prevent outflow of blood from the needle and to allow a guide wire to be inserted into the introducer needle in a bloodless seal-tight manner. Cope et al. US 2005/0033238 A1, provides a fluid passageway for withdrawing blood with re-sealing segments that permit passage of a wire guide therethrough and a needle hub assembly comprising a fluid-receiving chamber.

What is needed is an improved bloodless arterial percutaneous insertion system which prevents the escape of blood through the proximal end of a percutaneous insertion needle but more easily permits the clinician to readily identify that the needle end has entered the blood vessel.

### BRIEF DESCRIPTION OF THE DRAWINGS

FIG. 1 is a cross section view of the needle connector assembly illustrating the hemostatic end, the mail luer end, and the side tube, in accordance with a preferred embodiment.

FIG. 2 is a cross section view illustrating the connection of the needle connector through its male luer slip to a female luer needle hub, in accordance with a preferred embodiment.

FIG. 3 is an isometric assembled view of the needle connector assembly illustrating the connection of the male luer slip to a female luer needle hub, the guide wire entrance, and the side tube, in accordance with a preferred embodiment.

### DESCRIPTION OF THE PREFERRED EMBODIMENT

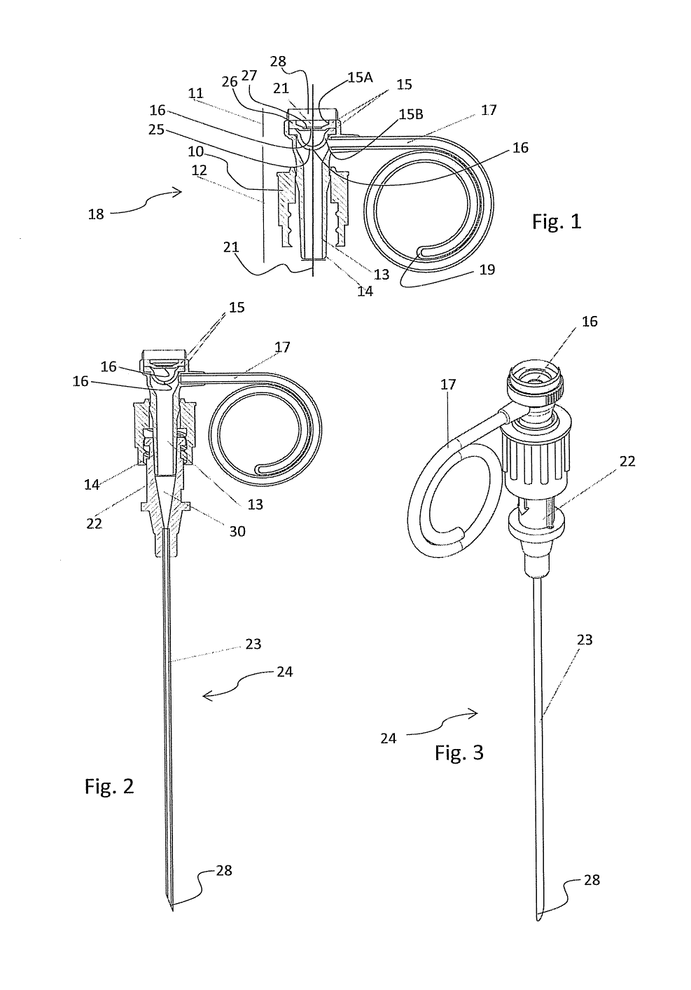

Referring to FIGS. 1-3, a bloodless arterial percutaneous insertion system 24 is shown. As used herein, the terms “a” or “an” shall mean one or more than one. The term “plurality” shall mean two or more than two. The term “another” is defined as a second or more. The terms “including” and/or “having” are open ended (e.g., comprising). The term “or” as used herein is to be interpreted as inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

Reference throughout this document to “one embodiment,” “certain embodiments,” “an embodiment,” or similar term means that a particular feature, structure, or characteristic described in connection with the embodiment is included in at least one embodiment of the present disclosure. Thus, the appearances of such phrases in various places throughout this specification are not necessarily all referring to the same embodiment. Furthermore, the particular features, structures, or characteristics may be combined in any suitable manner on one or more embodiments without limitation. The detailed description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention. In the description below, the term “distal” end of a component refers to the end of the described component that is in closest proximity to the patient when in use. The term “proximal” end of a component refers to the end of the component that is at the greatest distance from the patient when in use.

Referring to FIG. 1, a needle connector assembly 18 comprises a proximal end 11, a distal end 12, a housing 10, and a passageway 13 extending therebetween. The distal end 12 comprises a male luer slip 14 that can be coupled to a female luer 22 of a needle 23 (FIGS. 2 & 3) for percutaneous entry into a body vessel such as an artery.

Referring to FIG. 1, the proximal end 11 comprises a hemostatic segment 15 comprising an opening 16 permitting passage of a guidewire 21 therethrough. In the preferred embodiment, this hemostatic segment 15 comprises a soft elastomeric material such as silicone which permits the guidewire 21 to be inserted through the opening 16. The hemostatic segment 15 comprises a proximal portion 15A and a distal portion 15B. The proximal portion 15A generally comprises a perimeter ring 26 and a spanning member 27. The distal portion 15B comprises a bell or rounded configuration 25. Upon insertion of guidewire 21, the hemostatic segment 15 generally seals around the guidewire 21 such that blood or other fluids are prevented from entering the area 28 proximal to the hemostatic segment 15.

The distal end 11 is sized and configured for leak-free engagement with the proximal end 12 of the needle connector assembly 18. The needle connector assembly 18 comprises a side tube 17. The side tube 17 comprises a closed distal end 19 and is positioned between the proximal end 11 and the distal end 12 of the needle connector assembly 18. The side tube 17 is in fluid communication with the passageway 13 of the connector assembly 18. This arrangement permits the side tube 17 to receive blood or other fluids entering the passageway 13 of the connector assembly 18. Therefore, as shown in FIG. 2, the side tube 17 is adapted to receive the “squirt” or pulsating jets of arterial blood that results from a tip 28 of the needle 23 entering the vessel. In the preferred embodiment, the side tube 17 comprises a transparent surface such that a clinician can visualize fluid entering the tube 17. The side tube 17 is of a conventional and commercially available type, and may be formed from latex, silicone, polyvinyl chloride (PVC) or any other suitable material. In the preferred embodiment, the side tube 17 comprises a coiled configuration. This coiled configuration permits the tube 17 to be of longer length than if the tube 17 were of a straight design. Additionally, the coiled and transparent aspects of the tube 17 permit the clinician to more easily visualize the entry of blood within the tube 17 and the pulsating jets of, for example, arterial blood as it enters the system 18.

Referring to FIG. 2, the connector passageway 13 is aligned with the needle hub 22 and needle passageway 30 to form a path for insertion of the guidewire 21 into the body vessel. In this configuration, the clinician may readily insert the guidewire 21 through the opening 16 of hemostatic segment 15A, such that the guidewire 21 exits the opening 16 of the bell shaped 25 distal portion 15B, passes through needle passageway 30, through the needle 23 and into the body vessel. Upon insertion of guidewire 21, the hemostatic segment 15 seals around the guidewire 21 such that blood or other fluids are prevented from entering the area 28 proximal to the hemostatic segment 15, and instead, are directed into side tube 17.

A method of percutaneous insertion is provided. In a preferred embodiment, the method comprising the steps of providing an insertion assembly 24, the insertion assembly 24 comprising upper 18 and lower 22 members, said lower member 22 comprising a needle 23, said upper member 18 comprising a main chamber 13 and a side chamber 17, said upper 18 and lower 22 members being in fluid communication with each other; inserting said needle 23 into a blood vessel; 13 visualizing a return of blood into said side chamber 17; inserting a guidewire 21 through a hemostatic segment 15, said hemostatic segment 15 being positioned within said main chamber 13; moving said guidewire 21 through a passage comprised within said upper 18 and lower 22 members and said needle 23 into said blood vessel; and removing said insertion assembly 24 while the guidewire 21 remains in the blood vessel.

In one embodiment of the method, the hemostatic segment 15 further comprising a perimeter ring 26, a spanning member 27, and a rounded lower portion 25, said spanning member 27 extending between edges of said perimeter ring 26.

In one embodiment of the method, the hemostatic segment 15 comprises a through opening 16, said through opening 16 extending from said spanning member 27 to said rounded lower portion 25.

In one embodiment of the method, the hemostatic segment 15 comprises an elastomeric material, and is sealingly engaged with the guidewire 21 when the guidewire 21 is positioned within said hemostatic segment 15.

In one embodiment of the method, the upper member 18 comprises a housing 10, and the main chamber 13 is positioned within this housing 10, and the side tube 17 extends outward from the housing 10.

In one embodiment of the method, the side tube 17 comprises a coiled configuration.

The preferred materials of construction of the housing 10 are PVC and polycarbonate. The tube is preferably formed from PVC. The hemostatic segment 15 is preferably formed from silicone. The needle 23 is preferably stainless steel while its hub 22 is polypropylene, PVC or polycarbonate. However, while certain materials are discussed herein with respect to the components of the system 24, the system 24 is not limited to such materials. The system 24 may comprise polyurethane, silicone or like material (e.g., a soft plastic or elastomer). The examples and illustrations of the bloodless arterial percutaneous insertion system 24 are described herein with respect to a coiled transparent side tube 17. However, the inventive system 24 is equally applicable for use with side tubes 17 and chambers 17 of different configurations.

In the preferred embodiment of the bloodless arterial percutaneous insertion system 24, a luer connection between a male luer slip 14 that can be coupled to a female luer 22 of a needle 23 for percutaneous entry into a body artery is provided. Although the bloodless arterial percutaneous insertion system 24 of the preferred embodiment comprises a modified luer connector 14, 22, the connector 14, 22 need not be a luer type connector 14, 22. Rather, other connectors 14, 22 may be modified in accordance with this specification without departing from the scope and spirit of this disclosure. For example, in other embodiments, the connector 14, 22 may comprise barbs or ridges on an outer surface.

The foregoing disclosure and showings made in the drawing are merely illustrative of the principles of this invention and are not to be interpreted in a limiting sense. While the invention is shown in only a few forms, it is not just limited to the forms shown, but is susceptible to various changes and modifications without departing from the spirit thereof. The foregoing description of a preferred embodiment of the invention has been presented for the purpose of illustration and description. It is not intended to be exhaustive or to limit the invention to the precise form disclosed. Obvious modifications or variations are possible in light of the above teachings. The invention may be adapted for use in a number of environments.

The embodiment was chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention in accordance with the breadth of this disclosure and the appended claims, to which they are fairly, legally, and equitably entitled to be interpreted.

1. A percutaneous connection device comprising:
a housing and a side chamber;
the housing comprising first and second ends;
said housing further comprising a housing chamber, a connection member, and a hemostatic segment;
said housing chamber being in fluid communication with said side chamber;
said connection member defining said second end and adapted for coupling with a percutaneous insertion member; and
said hemostatic segment sealingly positioned proximate to the first end.

2. The percutaneous connection device of claim 1, the hemostatic segment comprising a through opening adapted for insertion of a guidewire.

3. The percutaneous connection device of claim 2, the hemostatic segment further comprising a perimeter ring and a spanning member, said spanning member extending between edges of said perimeter ring.

4. The percutaneous connection device of claim 3, the hemostatic segment comprising a rounded distal portion.

5. The percutaneous connection device of claim 2, the hemostatic segment comprising an elastomeric material.

6. The percutaneous connection device of claim 1, the side tube extending outward from said housing.

7. The percutaneous connection device of claim 6, the side tube comprising a coiled configuration.

8. A percutaneous connection system comprising:
upper and lower members;
said upper member comprising an upper housing and a side chamber;
the upper housing comprising first and second ends;
said upper housing further comprising an upper chamber, an upper connection member, and a hemostatic segment;
said upper chamber being in fluid communication with said side chamber;
said lower member comprising a lower housing comprising a lower chamber and a lower connection member;
said upper connection member defining said second end and adapted for coupling with said lower connection member; and
said hemostatic segment sealingly positioned proximate to the first end.

9. The percutaneous connection system of claim 8, the hemostatic segment comprising a through opening adapted for insertion of a guidewire.

10. The percutaneous connection system of claim 9, the hemostatic segment further comprising a perimeter ring and a spanning member, said spanning member extending between edges of said perimeter ring.

11. The percutaneous connection system of claim 10, the hemostatic segment comprising a rounded distal portion.

12. The percutaneous connection system of claim 9, the lower member comprising a needle member.

13. The percutaneous connection system of claim 8, the side tube extending outward from said housing.

14. The percutaneous connection system of claim 13, the side tube comprising a coiled configuration.

15. The percutaneous connection system of claim 14, the side tube comprising a closed side tube end.

16. A method of percutaneous insertion comprising the steps of:
providing an insertion assembly, said insertion assembly comprising upper and lower members, said lower member comprising a needle, said upper member comprising a main chamber and a side chamber, said upper and lower members being in fluid communication with each other;
inserting said needle into a blood vessel;
visualizing a return of blood into said side chamber;
inserting a guidewire through a hemostatic segment, said hemostatic segment being positioned within said main chamber;
moving said guidewire through a passage comprised within said upper and lower members and said needle into said blood vessel; and
removing said insertion assembly while the guidewire remains in the blood vessel.

17. The method of claim 16, the hemostatic segment further comprising a perimeter ring, a spanning member, and a rounded lower portion, said spanning member extending between edges of said perimeter ring.

18. The method of claim 17, the hemostatic segment comprising a through opening, said through opening extending from said spanning member to said rounded lower portion.

19. The method of claim 16, the hemostatic segment comprising an elastomeric material, the hemostatic segment sealingly with said guidewire when said guidewire is positioned within said hemostatic segment.

20. The method of claim 16, the upper member comprising a housing, said main chamber being positioned within said housing, said side tube extending outward from said housing.

21. The method of claim 20, the side tube comprising a coiled configuration.
